SEQUENCE LISTING (3 sequences)

SEQ ID NO: 1 - DNA, 18 bp, Artificial Sequence
  Sequence consensus
cacggtatagtctcgcca

SEQ ID NO: 2 - DNA, 23 bp, Artificial Sequence
  Sequence consensus
aggggctggggtgtggttctggc

SEQ ID NO: 3 - DNA, 15 bp, Artificial Sequence
  Sequence consensus
cccctaatcagtggc

(19) United States
(12) Patent Application Publication
(10) Pub. No.: US 2021/0024616 A1
(43) Pub. Date: Jan. 28, 2021

(54) Method for Purifying Antibodies from Raw Milk

(71) Applicant: Laboratoire Francais du Fractionnement et des Biotechnologies, Les Ulis (FR)

(72) Inventors: Philippe PAOLANTONACCI, Gif sur Yvette (FR); Beatrice CLAUDEL, Linas (FR); Abdessatar CHTOUROU, Elancourt (FR)

(73) Assignee: Laboratoire Francais du Fractionnement et des Biotechnolofies, Les Ulis (FR)

(21) Appl. No.: 16/956,845

(22) PCT Filed: Dec. 28, 2018
(86) PCT No.: PCT/EP2018/097077

§ 371 Date: Sep. 3, 2020

(30) Foreign Application Priority Data
Dec. 29, 2017 (FR) 1763368

(51) Int. Cl. C07K 16/04; C07K 1/36

(52) U.S. Cl. CPC C07K 16/04; C07K 1/36

(57) ABSTRACT

The present application relates to a method for preparing an antibody or antibody fragment composition from raw milk from a non-human mammal expressing said antibody or antibody fragment in its milk, comprising steps a) precipitation of the raw milk with caprylic acid, b) separation, consisting of centrifugation or filtration through a depth filter, and optionally c) filtration through an active carbon depth filter.

### FIELD OF THE INVENTION

The present invention is in the field of methods for purifying compositions comprising an antibody or antibody fragment produced in the milk of a non-human mammal. It relates to a process for preparing a composition comprising an antibody, an antibody fragment from raw milk of a non-human mammal expressing said antibody or antibody fragment in its milk, comprising a) a step of precipitating the raw milk with caprylic acid, b) a separation step consisting of centrifugation or filtration through a depth filter, and optionally c) a filtration step through an activated carbon depth filter.

### PRIOR ART

Antibodies are used in a large number of industrial and pharmaceutical applications, such as diagnostics and therapy. In order to obtain sufficient quantities on a regular basis, antibodies are generally recombinantly produced by expression systems, such as unicellular organisms (bacteria or yeasts), insect cells (baculovirus/insect cell system) or transgenic plants. However, these expression systems have many limitations, particularly in connection with imperfect protein folding, the impossibility of producing complex proteins such as antibodies or glycosylation that is incomplete or different from that found in humans.

Given these limitations, the expression systems that are the most commonly sed for the production of antibodies, particularly for pharmaceutical applications, are currently mammalian cells. As an example, the active ingredient in MabThera® (rituximab), a chimeric anti-CD20 antibody for the treatment of non-Hodgkin lymphoma, is recombinantly produced in the Chinese Ovary Hamster (CHO) cell line. This system allows for the production of antibodies with glycosylation patterns that are very close to those of the human endogenous proteins but generally offers low production yields. In addition, this system imposes significant production costs on manufacturers.

In order to produce antibodies with a high yield and at a lower cost than those obtained from cell lines, the expression of antibodies in the milk of non-human transgenic mammals, such as cows, rabbits or goats, has been developed. Indeed, it has been estimated that the gross cost of producing a recombinant protein in transgenic milk is 5 to 100-fold lower than the cost of producing it in the CHO cell line. In this approach, antibody expression is directed to the mammary epithelial cells. The antibody is thus secreted in the milk and can be recovered from this fluid by extraction and purification methods. As an example, the work of Wei et al., 2011, describing the expression of the chimeric antibody chHabl8 in the milk of transgenic mice, may notably be cited.

Although the production of antibodies in the milk of non-human mammals makes it possible to obtain a very satisfactory level of expression, the extraction and purification of antibodies from milk remains one of the limiting steps of this expression system.

Indeed, milk is a very complex biological fluid, constituted of about 10% by weight of dry matter and about 90% by weight of water and comprising various constituents that can be grouped into three categories. The first category, called lactoserum (or whey), is constituted of carbohydrates, soluble proteins, such as lactalbumins and lactoglobulins, as well as albumins and immunoglobulins originating in the blood, minerals and water-soluble vitamins. The second category, called the lipid phase (or cream), consists essentially of lipids in the form of an emulsion of fat globules of approximately 2 to 12 μm in diameter. The third category, called the colloidal micellar phase, consists essentially of casein proteins and calcium phosphate salts, which form colloidal micellar complexes capable of reaching diameters of approximately 0.5 μm, and is notably in the form of aggregates (“clusters”) of tricalcium phosphate.

Milk that has not been previously been subjected to a step of separation of one of its constituents is called “raw milk”. It includes all of the normal components of milk, in particular raw milk includes lipids and all proteins, whether they are present in whey or in the colloidal micellar phase (raw milk notably includes caseins and β-lactoglobulins).

The different constituents of milk can be separated according to several methods:
Skimming, in general by centrifugation, allows the “skim milk” (including lactoserum—also called “whey”—and caseins) to be separated from the cream (in other words the lipid phase). After removal of the cream (lipid phase), a “defatted raw milk” or “skim milk” is obtained, which includes all the proteins, regardless of whether they are present in the lactoserum or in the colloidal micellar phase (raw milk notably comprises caseins and β-lactoglobulins).
Clarification allows the lactoserum to be separated from the micellar phase (essentially casein proteins and calcium phosphate salts) and lipid (or cream) phase. According to the method used, clarification may also eliminate some lactoserum proteins by precipitation, for example by precipitation with citrate. “Clarified milk” (also called “lactoserum” or “whey”) is a clear milk that has lost its lipids (cream) and has already lost a portion of its proteins (including caseins, and in some cases, according to the method, certain lactoserum proteins).
Acidification, for example by adding lactic ferments or an acid such as acetic acid, makes it possible to precipitate the caseins present in the milk and thus to obtain lactoserum from skimmed milk.

The richness and complexity of each category of milk constituent makes it all the more difficult to perform a method of purifying an antibody or an antibody fragment.

Several patent applications describe methods of purifying antibodies from milk. For example, PCT application WO 97/12901 describes the purification of polyclonal antibodies from lactoserum of hyperimmunized animals (obtained after clarification of the raw milk). PCT application WO 2008/099077 discloses a method of purifying recombinant proteins, including antibodies, comprising steps of skimming and defatting, purification, elution and removal of milk proteins. PCT application WO 2016/156752 describes a purification method comprising a step of clarification with a poly(diallyldimethylammonium) salt followed by affinity chromatography and inactivation/elimination of pathogens, while PCT application WO 2016/034726 describes a purification method comprising steps of affinity chromatography, viral inactivation, cation exchange chromatography, anion exchange chromatography, and finally, nanofiltration. The steps of antibody purification from milk are generally numerous, and the methods of the prior art generally require a first step of “skimming”, in which fatty matter is separated from the milk, yielding two fractions: skim milk (including lactoserum and caseins) and cream. In addition, several downstream steps are generally necessary in order to obtain a composition comprising an antibody having a level of quality, purity, and sanitary safety that is deemed acceptable (e.g. filtration, chromatography, viral inactivation steps, etc.). These criteria are particularly important when a composition comprising an antibody is intended for a pharmaceutical application. Finally, if it is possible to precipitate caseins present in the milk by a simple acidification step (a phenomenon that makes it possible to obtain lactoserum), this type of precipitation (without caprylic acid) is insufficient as certain proteins, such as β-lactoglobulin, remain in the soluble fraction.

Patent applications describing methods of antibody purification comprising a precipitation with caprylic acid step, such as PCT applications WO 2006/064373, WO 2010/151632, and WO 2014/123485 exist. However, these applications concern raw materials other than milk, such as serum, cell culture medium, or cell lysates, and systematically comprise at least a first pre-purification step before the precipitation step with caprylic acid. As an example, plasma undergoes a first cryoprecipitation step, in order to recover cryosupernantant only, which may furthermore be treated with ethanol, before addition of caprylic acid.

There is, therefore, at present a need for new methods of purifying antibodies or antibody fragments from milk. In particular, there is a need for new methods that are simpler and faster, notably comprising fewer steps. There is also a need for new methods allowing the cost of purification to be reduced, preferably without impacting yield and/or quality of the purified antibody or antibody fragment. Finally, there is a need for novel methods from which a composition comprising an antibody or antibody fragment can be directly used, notably as a pharmaceutical product.

### SUMMARY OF THE INVENTION

The present invention relates to a method for preparing a composition comprising an antibody or an antibody fragment from raw milk of a non-human mammal comprising a step of precipitation of raw milk with caprylic acid.

Indeed, in the context of the present invention, the inventors have demonstrated that a method of preparing a composition comprising an antibody or antibody fragment from raw milk of a non-human mammal which comprises a first step of precipitation of the raw milk with caprylic acid makes it possible to simultaneously clarify, purify and secure said composition, despite the complexity of the raw material constituted by raw milk. The method of the invention is advantageous as it is very easy to implement—not only does it comprise few steps, it also does not require the implementation of a milk skimming step prior to the step of precipitation with caprylic acid. In addition, it allows for an excellent removal of undesired proteins, such as β-lactoglobulin. Indeed, the residual amount of this observed protein is lower with the method according to the invention than when a simple step of acidification with acetic acid is used. This method is also advantageous as it makes it possible to reduce the time necessary to purify an antibody or antibody fragment, without, however, reducing the quality or the quantity of the composition comprising the antibody or antibody fragment that is obtained. Purification cost is advantageously reduced, and profitability thus improved. The method of the invention also provides a composition comprising an antibody or antibody fragment suitable for use as a pharmaceutical product. As an example, the composition comprising an antibody or antibody fragment purified by the method of the invention may be administered to a subject, for example, orally, without undergoing additional purification or filtration/viral inactivation steps.

In a first aspect, the present invention therefore relates to a method of preparing a composition comprising an antibody or an antibody fragment, advantageously a monoclonal antibody or a monoclonal antibody fragment, from raw milk of a non-human mammal expressing said antibody or antibody fragment in its milk, comprising:
a) a step of precipitating raw milk with caprylic acid,
b) a separation step consisting of centrifugation or filtration through a depth filter, and optionally
c) a filtration step through an activated carbon depth filter.

Advantageously, step a) makes it possible both to clarify the milk and to secure it at a biological level and to purify the antibody or antibody fragment (i.e. to increase its proportion of dry weight in the solution obtained upon completion of step a) in relation to its proportion of dry weight in the raw milk).

Advantageously, step a) precipitates β-lactoglobulins.

Advantageously, the raw milk has not undergone any prior clarification and/or skimming and/or acidification step.

The separation (step b)) and filtration (step c)) steps of said method respectively allow the proteins precipitated by caprylic acid and lipids to be removed, and the caprylic acid itself to be eliminated.

Preferably, the total protein concentration of the raw milk prior to the step of precipitation with caprylic acid is comprised between 25 and 100 g/l, preferably between 30 and 60 g/l. According to a particularly preferred embodiment, the total protein concentration of the raw milk before the precipitation with caprylic acid of step a) is equal to 50 g/l.

Preferably, the concentration of antibody or antibody fragment of the raw milk before step a) of precipitation with caprylic acid is comprised between 3 and 50 g/l, more preferably between 5 and 30 g/l. According to an even more preferred embodiment, the concentration of antibody or antibody fragment of the raw milk before the step of precipitation with caprylic acid is equal to 20 g/l.

In certain embodiments, prior to step a) of precipitation with caprylic acid, the raw milk is not diluted or is diluted at a ratio (raw milk:diluent, expressed in volumes) ranging from 1:0.1 to 1:4. Preferably, to generate the solution before precipitation, raw milk is diluted to a ratio (raw milk:diluent, expressed in volumes) equal to 1:3.

Preferably, the final percentage (w/w) of caprylic acid in the raw milk (mass of caprylic acid/mass of raw milk×100) used in the precipitation step is comprised between 0.5 and 3.0%, more preferably between 1.0 and 2.5%. Even more preferably, the percentage (w/w) of caprylic acid used in the precipitation step is comprised between 1.3 and 2.0% and notably 1.7% or about 1.7%. (1.7±0.1%).

Preferably, after addition of caprylic acid to the raw milk in step a) of precipitation, the pH of the mixture is adjusted to a value of less than 4.8. More preferably, the pH of the mixture is adjusted to a value comprised between 4.0 and 4.8, even more preferably at a value of 4.3. The pH adjustment can be performed using any suitable acid, notably selected from acetic acid and citric acid. In some embodiments of the method according to the invention, the pH is adjusted by addition of acetic acid.

Preferably, step b) of separation is performed via a depth filtration step, which is preferably performed using a cellulose fiber-based filter. In a particular embodiment, the cut-off threshold of said filter is comprised between 10 and 80 μm, preferably between 20 and 50 μm. Preferably, the filter is a depth filter having a thickness of 4 to 5 mm, composed of cellulose fibers and perlite, with a cut-off threshold of between 10 and 50 μm, of the Seitz® T3500 type. In a preferred embodiment, the step of separation via a step of depth filtration is performed in the presence of a filter aid (used in alluvialization or as a precoat), which may be mineral (for example diatomaceous earth or perlite) or organic (such as cellulose).

The method according to the invention may further comprise at least one additional step, subsequent to step b) of centrifugation or filtration through a depth filter (when step c) is not implemented) or to step c) of filtration through an activated carbon depth filter (when this step is implemented), selected from the steps of:
Concentration, preferably by ultrafiltration and/or diafiltration,
Purification, preferably by chromatography, in particular by ion exchange or affinity chromatography, preferably chromatography on a cation exchange resin, chromatography on an anion exchange resin, or affinity chromatography, preferably affinity chromatography using aptamer ligands,
Formulation, and/or
Biological safety, preferably by the elimination and/or inactivation of residual pathogens, in particular viral inactivation and/or viral elimination.

Preferably, the antibody is an antibody having an isotype chosen from IgG and IgA, preferably IgG isotype. In a preferred embodiment, the purified IgG isotype antibody has conserved a distribution of IgG1, IgG2, IgG3 and IgG4 subclasses similar to that of the unpurified raw milk. When the antibody is a monoclonal antibody, it is preferably of IgG isotype, and in particular of IgG1 isotype. Among the antibody fragments comprising a constant domain, said constant domain comprising an Fc fragment capable of binding to FcR receptors, or the antibody fragments comprising an Fc fragment capable of binding to FcR receptors, the Fc fragment is preferably of an isotype selected from IgG and IgA, preferably isotype IgG. In a preferred embodiment, the antibody fragment comprising a constant domain, said constant domain comprising an Fc fragment capable of binding to FcR receptors, or the antibody fragment comprising an Fc fragment capable of binding to FcR receptors maintains, after purification, a distribution of the IgG1, IgG2, IgG3 and IgG4 subclasses similar to that of unpurified raw milk. When the antibody is a monoclonal antibody fragment comprising a constant domain, said constant domain comprising an Fc fragment capable of binding to FcR receptors, or an antibody fragment comprising an Fc fragment capable of binding to FcR receptors it is preferably of IgG isotype, and in particular of IgG1 isotype.

Preferably, the non-human mammal expressing an antibody or antibody fragment in its milk is a rabbit, cow or goat.

### DESCRIPTION OF THE FIGURES

FIG. 1. Diagram illustrating the method according to the invention.

### DETAILED DESCRIPTION OF THE INVENTION

As indicated previously, in the context of the present invention, the inventors have demonstrated a new method of preparing a composition comprising an antibody or antibody fragment from raw milk of a non-human mammal expressing said antibody or antibody fragment in its milk and comprising a step of precipitating the raw milk with caprylic acid. Indeed, the inventors have surprisingly demonstrated that the step of precipitating the raw milk with caprylic acid makes it possible to simultaneously clarify, purify and secure an antibody or antibody fragment purified from the raw milk with this single step. Thus, said method comprising a step of precipitation with caprylic acid is referred to herein as a “three-in-one” method, as it fulfills three functions that have heretofore been performed by individual steps. The inventors have notably demonstrated that the step of precipitating the raw milk with caprylic acid makes it possible to clarify the milk, purifying the antibody or antibody fragment present in the milk by precipitating other proteins, including proteins of the casein family (but also β-lactoglobulin, lactoferrin, serum albumin, a-lactalbumin), and improving the safety of the product by inactivation and elimination of viruses by at least 4 decimal logs. Particularly advantageously, the inventors have demonstrated that the step of precipitation with caprylic acid allows for precipitation of β-lactoglobulin.

Preparation Method

A first aspect of the invention therefore relates to a method of preparing a composition comprising an antibody or antibody fragment from raw milk of a non-human mammal expressing said antibody or antibody fragment in its milk comprising:
a) a step of precipitation of the raw milk with caprylic acid,
b) a separation step consisting of centrifugation or filtration through a depth filter, and optionally
c) a filtration step through an activated carbon depth filter.

Advantageously, step a) makes it possible to simultaneously clarify the milk and render it biologically safe and to purify the antibody or antibody fragment (i.e. increasing its proportion of dry weight in the solution obtained upon completion of step a) in relation to its proportion of dry weight in the raw milk).

Steps a) to c) are implemented in the following order: a), then b), and then optionally c). In some cases (see below), additional steps can be interposed:
Before step a) (excluding any step of prior separation of one of the constituents of raw milk such as clarification, skimming or acidification).
   For example, step a) may be preceded by a step of freezing and then thawing the raw milk and/or by a step of diluting the raw milk, preferably in water.
Between steps a) and b).
   For example, an incubation step may be added between steps a) and b).
Between steps b) and c).
   For example, an incubation step may be added between steps b) and c).
After step b) of centrifugation or filtration through a depth filter (when step c) is not implemented) or after step c) of filtration through an activated carbon depth filter (when step c) is implemented).
   For example, steps of purification, concentration, formulation, and/or biologically securing the composition may be added after step b) or step c).

These various additional steps are detailed below.

The purification process according to the invention is preferably performed using raw milk of a non-human mammal, preferably a transgenic non-human mammal, comprising the antibody or antibody fragment in unpurified form, that is to say further comprising other contaminating products (other proteins, DNA, sugars, lipids, etc.).

Milk

In the context of the present invention, “milk” refers to a milk obtained from a transgenic or non-transgenic non-human mammal (referred to as a natural non-human mammal). “Transgenic milk” refers to a milk obtained from a transgenic non-human mammal, that is to say from a non-human mammal that has been genetically modified such that it produces an recombinant antibody or antibody fragment of interest in its milk. “Natural milk” or “non-transgenic milk” refers to milk obtained from a non-transgenic non-human mammal.

A natural (non-transgenic) non-human mammal can notably be hyperimmunized to increase the amount of a polyclonal antibody directed against a particular antigen present in its milk.

A transgenic non-human mammal may be obtained by direct injection of the gene(s) of interest (here, the rearranged genes encoding the heavy and light chains of the antibody or the antibody fragment) into a fertilized egg (Gordon et al., 1980). A transgenic non-human mammal may also be obtained by introducing the gene(s) of interest (here, the rearranged genes encoding the heavy and light chains of the antibody or the antibody fragment) into an embryonic stem cell and preparation of the mammal by a chimera aggregation method or a chimera injection method (see Manipulating the Mouse Embryo, A Laboratory Manual, Second edition, Cold Spring Harbor Laboratory Press (1994); Gene Targeting, Practical Approach, IRL Press at Oxford University Press (1993)). A transgenic non-human mammal can also be obtained by a cloning technique in which a nucleus, in which the gene(s) of interest (here, the rearranged genes encoding the heavy and light chains of the antibody or for the antibody fragment) have been introduced, is transplanted into an enucleated egg (Ryan et al., 1997, Cibelli et al., 1998, WO0026357A2). A transgenic non-human mammal producing an antibody or antibody fragment of interest can be prepared by the above methods. The antibody or antibody fragment can then be accumulated in the transgenic non-human mammal and harvested, notably from the milk of the mammal. For the production of proteins, in particular antibodies or antibody fragments, in the milk of transgenic non-human mammals, preparation methods are notably described in WO9004036A1, WO9517085A1, WO0126455A1, WO2004050847A2, WO2005033281A2, WO2007048077A2. In at least some of these methods, the sequence encoding the antibody or antibody fragment is operably linked to a control sequence that allows the coding sequence to be expressed in the milk of a transgenic non-human mammal. The coding sequence may be operably linked to a control sequence that allows the coding sequence to be expressed in the milk of a transgenic non-human mammal.

A DNA sequence that is suitable for directing production in the milk of transgenic animals may carry a 5′ promoter region derived from a protein naturally present in milk. Such a promoter is therefore under the control of hormonal and tissue factors and is particularly active in lactating breast tissue. The promoter may further be operably linked to a DNA sequence directing the production of a signal sequence that directs the secretion of the transgene protein through the mammary epithelium into the milk. In some embodiments, a 3′ sequence, which can be derived from a protein naturally present in the milk, can be added to enhance mRNA stability. As used herein, a “signal sequence” is a nucleic acid sequence that encodes a protein secretion signal and, when operably linked downstream of a nucleic acid molecule encoding a transgenic protein, directs its secretion. The signal sequence can be a native human signal sequence, an artificial signal sequence, or can be obtained from the same gene as the promoter used to direct the transcription of the sequence coding the antibody or antibody fragment, or from another protein normally secreted from a mammalian mammary epithelial cell. In some embodiments, the promoters may be milk specific promoters. As used herein, a “milk specific promoter” is a promoter that naturally directs the expression of a gene in a cell that secretes a protein into milk (e.g., a mammary epithelial cell), which comprises, e.g., casein promoters (for example alpha, notably alpha S1 or alpha S2; beta, gamma, or kappa), the whey acid protein (WAP) promoter, the β-lactoglobulin promoter, and the alpha-lactalbumin promoter. Also included in this definition are promoters that are specifically activated in mammary tissue, such as e.g. the Long Terminal Repeat (LTR) promoter of the mouse mammary tumor virus (MMTV).

Non-human mammals of particular interest include goats, sheep, bovines (including cows), camels, llamas, mice, rats, and rabbits. According to a preferred embodiment of the invention, the non-human mammal expressing an antibody or antibody fragment in its milk is a bovine, preferably a cow, a goat or a rabbit.

“Raw milk” more particularly refers to a milk that has undergone neither a prior step of separation of one of its constituents nor a purification step intended to increase the relative proportion of the antibody or antibody fragment in relation to the other milk constituents. In particular, within the context of the invention, “raw milk” has not undergone any skimming and/or clarification and/or defatting and/or acidification step before the step of precipitation with caprylic acid. Advantageously, the raw milk is therefore an unclarified milk comprising all of the constituents initially present in said milk (lipids, proteins, carbohydrates, minerals, vitamins, etc.). In particular, raw milk includes lipids and all proteins (notably caseins and β-lactoglobulins). “Raw milk” within the context of the invention comprises a milk having optionally undergone one or more treatment steps other than steps of prior separation of one of its constituents or of purification. Thus, “raw milk” within the context of the invention comprises a milk which has optionally undergone freezing/thawing, e.g. in the case of prior storage of the milk, before the step of precipitation with caprylic acid. “Raw milk” within the context of the invention also comprises milk which has optionally been diluted. Indeed, such steps are not steps of prior separation of one of its components nor purification. Preferably, the raw milk has not undergone any other treatment than a freezing/thawing and/or a dilution prior to step a) of precipitation with caprylic acid.

The term “raw milk” comprises “transgenic raw milk” derived from transgenic non-human mammals, and “natural raw milk” derived from non-transgenic non-human mammals; preferably, the raw milk is transgenic raw milk.

“Skimming” milk refers to a step of lipid (also called “cream”) removal, which leads to “skim milk” (also called “defatted milk”) including lactoserum (or “whey”) and the colloidal phase (comprising caseins). “Skim milk” or “defatted milk” therefore comprises lactoserum (or “whey”) and all proteins, whether they are present in the lactoserum or in the colloidal micellar phase, and notably caseins and lactoserum proteins, as well as any proteins of interest, such as antibodies or antibody fragments.

“Milk clarification” refers to a step that separates the lactoserum from micellar and lipid phases. Clarification can be performed in various ways, and in particular by centrifugation, filtration, or acidification. Depending on the method used, clarification may also eliminate some lactoserum proteins by precipitation, for example by precipitation with citrate. “Clarified milk” or “lactoserum” or “whey” is a clear milk that has already lost its lipids (cream) and that has lost a portion of its proteins (including caseins, and in some cases, according to the method, some lactoserum proteins).

“Milk acidification” refers to a step which allows caseins proteins present in the milk to be precipitated and thus to obtain lactoserum from skim milk, e.g. by adding lactic ferments or an acid such as acetic acid.

“Whey” or “lactoserum ” or “clarified milk” refers to a milk that has undergone one or more steps leading to the elimination of lipids and caseins, e.g. a clarification step by acid precipitation of caseins.

Preferably, the total protein concentration of the raw milk before step a) of precipitation with caprylic acid is comprised between 25 and 100 g/l. Preferably, the raw milk has a total protein concentration between 25 and 90 g/l, between 25 and 80 g/l, between 25 and 75 g/l, between 25 and 70 g/l, between 25 and 60 g/L, between 25 and 50 g/l, more preferably between 30 and 90 g/l, between 30 and 80 g/l, between 30 and 70 g/l, between 30 and 60 g/l, between 30 and 50 g/l, more preferably between 40 and 90 g/l, between 40 and 80 g/l, between 40 and 70 g/l, between 40 and 60 g/l, between 40 and 55 g/l, and in particular between 45 and 55 g/l. Even more preferably, the raw milk has a total protein concentration of 50 g/l. The total protein concentration of the raw milk may be determined by the skilled person in view of his general knowledge. As non-limiting examples, the total protein concentration can be determined by total protein assay techniques such as the Biuret method, BCA (protein assay with bicinchoninic acid), Bradford, Coomassie blue, determination of organic nitrogen according to Kjeldahl, UV or IR absorption, preferably by the BCA assay.

“Total proteins” represent all of the proteins of the composition and comprise the antibody or antibody fragment to be purified as well as the contaminating proteins, in particular caseins and lactoserum proteins such as β-lactoglobulin. As indicated above, “raw milk” within the context of the invention comprises a raw milk having optionally undergone a dilution step prior to the step of precipitation with caprylic acid. According to a first embodiment, the raw milk is not diluted. According to a second embodiment, and in particular when the total protein concentration and/or the antibody or antibody fragment concentration is too high, the raw milk is diluted in a diluent such as water or a buffer solution. Preferably, the diluent is purified water. Preferably, the ratio by volume of raw milk to diluent (raw milk : diluent) is between 1:0.1 and 1:4. Preferably, the ratio by volume of raw milk on diluent (raw milk:diluent) is comprised between 1:0.1 and 1:3.9, between 1:0.2 and 1:3.8, between 1:0.4 and 1:3.7, between 1:0.6 and 1:3.6, between 1:0.8 and 1:3.5, between 1:0.9 and 1:3.4, between 1:1 and 1:3.3, between 1:1.1 and 1:3.2, between 1:1.2 and 1:3.2, between 1:1.1 and 1:3.1, or between 1:1.5 and 1:3.5, between 1:2 and 1:3.5, between 1:2.5 and 1:3.5. Even more preferably, the ratio by volume of raw milk to diluent (raw milk:diluent) is equal to 1:3.

Antibody and Antibody Fragment

“Antibody” or “immunoglobulin” refers to a molecule comprising at least one domain binding to a given antigen and a constant domain comprising an Fc fragment capable of binding to FcR receptors. By “antibody fragment” is meant a functional part of an antibody such as a domain binding to a given antigen or a constant domain comprising an Fc fragment capable of binding to FcR receptors.

In most mammals, such as humans and mice, an antibody is composed of 4 polypeptide chains: 2 heavy chains and 2 light chains linked to one another by a variable number of disulfide bridges providing flexibility to the molecule. Each light chain consists of a constant domain (CL) and a variable domain (VL); the heavy chains being composed of a variable domain (VH) and 3 or 4 constant domains (CH1 to CH3 or CH1 to CH4) according to antibody isotype. In a few rare mammals, such as camels and llamas, antibodies consist of only two heavy chains, each heavy chain comprising a variable domain (VH) and a constant region.

Variable domains are involved in antigen recognition, while constant domains are involved in the biological, pharmacokinetic and effector properties of the antibody.

Unlike the variable domains whose sequence varies widely from one antibody to another, the constant domains are characterized by an amino acid sequence that is very similar from one antibody to another, characteristic of the species and the isotype, eventually with a few somatic mutations. The Fc fragment is naturally composed of the constant region of the heavy chain excluding the CH1 domain, that is to say the lower hinge region and constant domains CH2 and CH3 or CH2 to CH4 (according to the isotype). In human IgG1, the complete Fc fragment is composed of the C-terminal portion of the heavy chain from the cysteine residue at position 226 (C226), the numbering of amino acid residues in the Fc fragment being that of the EU index described in Edelman et al., 1969 and Kabat et al., 1991 throughout the present description. The corresponding Fc fragments of other types of immunoglobulins can be easily identified by the skilled person by sequence alignments.

The Fcγ fragment is glycosylated at the CH2 domain with the presence, on each of the 2 heavy chains, of an N-glycan linked to the asparagine residue at position 297 (Asn 297).

The following binding domains, located in the Fcγ, are important for the biological properties of the antibody:
FcRn receptor binding domain, involved in the pharmacokinetic properties (in vivo half-life) of the antibody:
Different data suggest that certain residues located at the interface of the CH2 and CH3 domains are involved in FcRn receptor binding.
C1q complement protein binding domain, involved in the CDC (for “complement dependent cytotoxicity”) response: located in the CH2 domain;
FcR receptor binding domain, involved in phagocytosis or ADCC (for “antibody-dependent cellular cytotoxicity) type responses: located in the CH2 domain.

Within the context of the invention, the Fc fragment of an antibody may be natural, as defined above, or may have been modified in various ways. The modifications may include the deletion of certain portions of the Fc fragment and/or different amino acid substitutions that may affect the biological properties of the antibody. In particular, when the antibody is an IgG, it may comprise mutations intended to increase binding to the FcγRIII (CD16) receptor, as described in WO00/42072, Shields et al., 2001, Lazar et al., 2006, WO2004/029207, WO2004/063351, WO2004/074455. Mutations allowing increased FcRn receptor binding and thus in vivo half-life may also be present, as described for example in Shields et al., 2001, Dall'Acqua et al., 2002, Hinton et al., 2004, Dall'Acqua et al., 2006(a), WO00/42072, WO02/060919A2, WO2010/045193, or WO2010/106180A2. Other mutations, such as those allowing decreased or increased complement protein binding and thus the CDC response, may or may not be present (see WO99/51642, WO2004/074455A2, Idusogie et al., 2001, Dall'Acqua et al., 2006(b) and Moore et al., 2010).

The antibody or antibody fragment produced in the milk subjected to the purification process according to the invention may be recombinant (when coded by a heterologous sequence inserted in the genome of a transgenic non-human animal) or non-recombinant (when coded by one or more sequences naturally produced by the non-human, potentially hyperimmunized, animal). In the case of a recombinant antibody, the antibody produced by the transgenic non-human animal is monoclonal. However, in the case of a non-recombinant antibody, the antibody produced by the non-transgenic non-human animal (in particular hyperimmunized against a given antigen) will be a monospecific or polyspecific polyclonal type-antibody. In a preferred embodiment, the antibody purified in the context of the purification process according to the invention is a monoclonal antibody. In a particular embodiment, the antibody fragment purified in the context of the purification process according to the invention is a monoclonal antibody fragment. In a particular embodiment, the antibody fragment comprises a constant domain, said constant domain comprising an Fc fragment capable of binding to FcR receptors, preferably the antibody fragment comprises a monoclonal antibody constant domain comprising an Fc fragment capable of binding to FcR receptors. In a particular embodiment, the antibody fragment comprises an Fc fragment, preferably a monoclonal antibody Fc fragment. In a particular embodiment, the antibody fragment comprises a given antigen binding domain, preferably a monoclonal antibody binding domain.

“Monospecific polyclonal antibody” or “monospecific polyclonal antibody composition” refers to a composition comprising antibody molecules directed against the same antigen, but produced by several B lymphocyte clones stimulated during immunization with the antigen. A monospecific polyclonal antibody thus groups several monoclonal antibodies directed against the same antigen and produced by distinct B lymphocyte clones. The different monoclonal antibodies included in the monospecific polyclonal antibody may be directed against different epitopes (or antigenic portion) of the same antigen.

“Polyspecific polyclonal antibody” or “polyspecific polyclonal antibody composition refers to a composition comprising antibody molecules directed against different antigens and produced by several B lymphocyte clones stimulated upon encountering one of the antigens. The collection of antibodies present in the milk of a transgenic non-human animal is an example of a polyspecific polyclonal antibody.

“Monoclonal antibody” or “monoclonal antibody composition” refers to a composition comprising antibody molecules having identical and unique antigenic specificity. The antibody molecules present in the composition may vary in their post-translational modifications, and in particular in their glycosylation structures or their isoelectric point, but have all been coded by the same heavy and light chain sequences and therefore, before any post-translational modification, have the same protein sequence. Some differences in protein sequence, related to post-translational modifications (such as e.g. cleavage of C-terminal lysine of the heavy chain, the deamidation of asparagine residues and/or the isomerization of aspartate residues), may nevertheless exist between the different antibody molecules present in the composition.

The monoclonal antibody or monoclonal antibody fragment purified in the context of the invention may preferably be chimeric, humanized, or human.

“Chimeric” antibody refers to an antibody which contains a naturally occurring variable region (light chain and heavy chain) derived from an antibody of a given species in combination with the constant regions of the light chain and heavy chain of an antibody of a species that is heterologous to said given species. Advantageously, if the monoclonal antibody composition for use as a medicament according to the invention comprises a chimeric monoclonal antibody, the latter comprises human constant regions. Starting from a non-human antibody, a chimeric antibody can be prepared using genetic recombination techniques well-known to the skilled person. For example, the chimeric antibody may be made by cloning, for the heavy chain and the light chain, a recombinant DNA comprising a promoter and a sequence coding for the variable region of the non-human antibody, and a sequence coding for the constant region of a human antibody. For methods of preparing chimeric antibodies, reference may be made e.g. to Verhoeyn et al., 1988.

“Humanized” antibody refers to an antibody which contains CDR regions derived from an antibody of non-human origin, the other parts of the antibody molecule being derived from one (or more) human antibodies. In addition, certain residues of the framework regions (called FRs) may be modified to maintain binding affinity (Jones et al., 1986; Verhoeyen et al., 1988; Riechmann et al., 1988). The humanized antibodies according to the invention may be prepared by techniques known to the skilled person such as CDR grafting, resurfacing, superhumanization, human string content, FR libraries, guided selection, FR shuffling and humaneering, as summarized in the review by Almagro et al., 2008.

A “human” antibody refers to an antibody whose entire sequence is of human origin, i.e. whose coding sequences have been produced by recombination of human genes coding the antibodies. Indeed, it is now possible to produce transgenic animals (e.g. mice) that are capable, upon immunization, of producing a complete repertoire of human antibodies in the absence of endogenous immunoglobulin production (see Jakobovits et al., 1993(a); Jakobovits et al., 1993(b); Bruggermann et al., 1993; Duchosal et al., 1992; U.S. Pat. Nos. 5,591,669; 5,598,369; 5,545,806; 5,545,807; 6,150,584). Human antibodies may also be obtained from phage display libraries (Hoogenboom et al., 1991; Marks et al., 1991; Vaughan et al., 1996).

The antibodies may be of several isotypes, depending on the nature of their constant region: the constant regions γ, α, μ, ε and δ respectively correspond to immunoglobulins IgG, IgA, IgM and IgD.

The antibody advantageously purified in the context of the invention may preferably be of IgG, IgA, IgM or IgD isotype, preferably according to the proportions present in the raw milk. When the antibody is monoclonal, it will in principle be a single isotype. In one embodiment, the antibody (notably monoclonal) purified in the context of the invention is of IgA isotype. Advantageously, the antibody (notably monoclonal) purified in the context of the invention is of IgG isotype. Indeed, the IgG isotype shows an ability to generate ADCC (“Antibody-Dependent Cellular Cytotoxicity”) activity in the largest number of individuals (humans) and is thus chiefly used for pharmaceutical applications of monoclonal antibodies.

The y constant regions comprise several subtypes: γ1, γ2, γ3, these three types of constant regions having the characteristic of binding human complement, and y4, thus creating the IgG1, IgG2, IgG3, and IgG4 subisotypes. Preferably, the antibody purified by the method of the invention is of IgG1, IgG2, IgG3 and/or IgG4 isotype. Advantageously, the proportion of each IgG subisotype present in the raw milk is conserved at the end of the process according to the invention. When the antibody is monoclonal, it will in principle be of a single isotype, and may in particular be of IgG1 isotype, the most used for monoclonal antibodies for therapeutic purposes.

When the monoclonal antibody fragment purified in the context of the invention comprises a constant domain, said constant domain comprising an Fc fragment capable of binding to FcR receptors, or comprises an Fc fragment capable of binding to FcR receptors, this will advantageously be of an isotype selected from IgG and IgA, preferably of isotype IgG, and in particular IgG1.

As a non-limiting example of antibodies (monoclonal or polyclonal, preferably monoclonal) that are of interest to express in a transgenic non-human mammal, mention may be made of an antibody directed against one of the following antigens:
Rhesus D, anti-Rhesus D antibodies being useful for the prevention of alloimmunisation in Rhesus-negative individuals,
Antigens expressed by cancer cells, that may be targeted in the treatment of cancers, and in particular: CD20, Her2/neu, CD52, EGFR, EPCAM, CCR4, CTLA-4 (CD152), CD19, CD22, CD3, CD30 , CD33, CD4, CD40, CD51 (Integrin alpha-V), CD80, CEA, FR-alpha, GD2, GD3, HLA-DR, IGF1R (CD221), phosphatidylserine, SLAMF7 (CD319), TRAIL-R1, TRAIL-R2.
Antigens expressed by cells infected with pathogenic agents, that may be targeted in the treatment of infection by pathogenic agents, and in particular: Clostridium difficile antigens, Staphylococcus aureus antigens (notably CIfA and lipotheicoic acid), cytomegalovirus antigens (notably glycoprotein B), Escherichia coli antigens (notably Shiga-like toxin, IIB subunit), respiratory syncytial virus antigens (Protein F notably), hepatitis B virus antigens, influenza A virus antigens (notably haemagglutinin), Pseudomonas aeruginosa serotype IATS 011 antigens, rabies virus antigens (notably glycoprotein), phosphatidylserine.
Antigens expressed by immune cells, that may be targeted in the treatment of autoimmune diseases, and in particular: CD20, CD52, CD25, CD2, CD22, CD3, and CD4.
Anti-cytokine antigens, and in particular anti-TNFα

Advantageously, the antibody (monoclonal or polyclonal, preferably monoclonal) purified from the raw milk is chosen from the antibodies directed against the following antigens: Rhesus D, CD2, CD3, CD4, CD19, CD20, CD22, CD25, CD30, CD33, CD40, CD51 (Integrin alpha-V), CD52, CD80, CTLA-4 (CD152), SLAMF7 (CD319), Her2/neu, EGFR, EPCAM, CCR4, CEA, FR-alpha, GD2, GD3, HLA-DR, IGF1R (CD221), phosphatidylserine, TNFα, TRAIL-R1, TRAIL-R2, Clostridium difficile antigens, Staphylococcus aureus antigens, cytomegalovirus antigens, Escherichia coli antigens, respiratory syncytial virus antigens, hepatitis B antigens, influenza A virus antigens, Pseudomonas aeruginosa serotype IATS O11 antigens, rabies virus antigens, or phosphatidylserine.

When an antibody fragment comprising a given antigen binding domain, preferably a monoclonal antibody binding domain, is purified in the context of the invention, it will also advantageously be directed against one of the above-indicated antigens.

Preferably, the antibody or antibody fragment concentration of the raw milk before step a) of precipitation with caprylic acid is comprised between 3 and 50 g/l. Preferably, the raw milk has an antibody or antibody fragment concentration between 3 and 45 g/l, between 3 and 40 g/l, between 3 and 35 g/l, between 3 and 30 g/l, between 3 and 25 g/l, between 5 and 45 g/l, between 5 and 40 g/l, between 5 and 35 g/l, between 5 and 30 g/l, between 5 and 25 g/l, between 10 and 45, between 10 and 40 g/l, between 10 and 35 g/l, between 10 and 30 g/l, between 10 and 25 g/l, between 15 and 45, between 15 and 40 g/l, between 15 and 35 g/l, between 15 and 30 g/l, between 315 and 25 g/l, between 16 and 24 g/l, between 17 and 23 g/l, between 18 and 22 g/l, or between 19 and 21 g/l. Even more preferably, the concentration of antibody or antibody fragment of the raw milk is equal to 4 g/l, 5 g/l, 6 g/l, 7 g/l, 8 g/l, 9 g/l, 10 g/l, 11 g/l, 12 g/l, 13 g/l, 14 g/l, 15 g/l, 16 g/l, 17 g/l, 18 g/l, 19 g/l, 20 g/l, 21 g/l, 22 g/l, 23 g/l, 24 g/l, or 25 g/l. The antibody or antibody fragment concentration of the raw milk can be determined by the skilled person in view of their general knowledge. As a non-limiting example, the antibody or antibody fragment concentration can be determined by ELISA, EIA, RIA, nephelometry, radial immunodiffusion (Mancini et al., 1965) or immunosensor (Campanella et al., 2009). Preferably, the antibody or antibody fragment concentration of the raw milk is determined by an ELISA test.

Step a)

Step a) of the method according to the invention is a step of precipitation with caprylic acid. This step makes it possible simultaneously clarify the raw milk (possibly frozen and then thawed and/or diluted), to very substantially purify the antibody or antibody fragment, and to inactivate/eliminate pathogens (thereby improving the biological safety of the composition). In other words, step a) amounts to combining, in a single step, the equivalent of a purification step, a clarification step and a biological safety step. In this step, caprylic acid is mixed with the raw milk (possibly frozen and then thawed and/or diluted). It should be noted that this step allows for a purification that is much more effective than a simple step of acidification with acetic acid, which does not sufficiently reduce the amount of certain milk proteins, such as β-lactoglobulin. Thus, step a) of the method is highly advantageous as it makes it possible to precipitate β-lactoglobulins.

Caprylic acid, of formula C₈H₁₆O₂, is a linear saturated fatty acid chain also known as 1-heptanecarboxylic, octanoic, octoic, or octic acid (see also CAS Reg. No. 124-07-2 and U.S. Pat. Nos. 2,821,534 and 3,053,869). By “caprylic acid” is meant herein caprylic acid in acid form as well as in the form of caprylate salt, such as sodium caprylate or potassium caprylate. Any caprylate salt can nevertheless be considered. Preferably, said caprylate salt is a pharmaceutically acceptable salt. Preferably, the caprylic acid used in step a) is in acid form.

Preferably, the final percentage (w/w) of caprylic acid relative to the raw milk (mass of caprylic acid/mass of raw milk×100) used in step a) is between 0.5 and 3.0%, between 0.6 and 2.9%, between 0.7 and 2.8%, between 0.8 and 2.7%, between 0.9 and 2.6%, between 1 and 2.5%, between 1 and 2.4%, between 1 and 2.3%, between 1 and 2.2%, between 1 and 2.1%, between 1.3 and 3%, between 1.3 and 2.5% between 1.3 and 2.2% between 1.3 and 2%, more preferably between 0.5 and 2.5%, between 1 and 2.5%, between 1 and 2%, between 1.5 and 2.0%. Even more preferably, the percentage (w/w) of caprylic acid relative to the milk used in step a) is comprised between 1.0 and 2.5%, preferably between 1.3 and 2.0%, and notably 1.7% or about 1.7% (1.7±0.1%).

According to a first embodiment, the caprylic acid is added all at once. According to a second embodiment, the caprylic acid is added over several times, in particular twice. In the context of the present invention, caprylic acid is advantageously added all at once.

According to a first embodiment, caprylic acid is added over a period of time of less than 10 minutes, preferably less than 5 minutes.

In a particular embodiment of the invention, caprylic acid is left in contact over a period of time greater than 5 minutes, greater than 10 minutes, preferably greater than 30 minutes, preferably between 1 and 2 hours.

After adding caprylic acid to the raw milk in step a), said composition is referred to herein as “mixture”.

According to a particular embodiment of the invention, the pH of the mixture is preferably adjusted by adding a suitable acid, in particular chosen from acetic acid and citric acid. In some embodiments of the method according to the invention, the pH is adjusted by adding a strong acid, optionally diluted. In one embodiment of the method according to the invention, the pH is adjusted by addition of acetic acid. Decreasing the pH is important to ensure precipitation and also advantageously improves the biological safety of the mixture, as some viruses are inactivated at acidic pH. According to a particular embodiment of the invention, after addition of the caprylic acid in step a) but before any other step (e.g. incubation or filtration), the pH of the mixture is advantageously adjusted to a value of less than 5, more preferably less than 4.8.

According to a particular embodiment of the invention, the pH of the mixture is preferably adjusted to a value comprised between 4.0 and 5.0, between 4.1 and 4.9, between 4.2 and 4.8, and between 4.2 and 4.7, more preferably between 4.2 and 4.6, between 4.2 and 4.6, or between 4.3 and 4.6. According to a particular embodiment of the invention, after the addition of caprylic acid in step a), the pH of the mixture is preferably adjusted to a value of 4.3. This value is optimal for the precipitation of elements of the milk that are considered as unwanted contaminants of the antibody or antibody fragment and thus for the purification of the antibody or antibody fragment, while a value of 4.6 is optimal for viral inactivation. Depending on which aspect to be favored, the skilled person may choose a final pH value in the ranges indicated above and in particular between 4.0 and 4.8.

Advantageously, step a) of precipitation allows for the formation of a precipitate comprising caseins as well as other unwanted milk proteins such as β-lactoglobulin, lipids, evenentually caprylic acid and certain pathogens.

Advantageously, step a) of precipitation also makes it possible to inactivate and/or eliminate pathogens, and in particular viruses that may be present in the raw milk prior to the implementation of step b) of separation and without the addition of other compounds to the mixture. In particular, the inventors have demonstrated that non-enveloped viruses, such as PPV, are precipitated by caprylic acid while enveloped viruses, such as X-MLV, are inactivated. Thus, the method according to the invention makes it possible to reduce the infectious viral titer by more than 4 decimal logs for each of these types of virus.

Advantageously, step a) of precipitation makes it possible to reduce the amount of infectious enveloped-type viruses (X-MLV for example) and/or the amount of infectious non-enveloped-type viruses (PPV for example) that may be present in the raw milk by at least 4 logs (logarithms decimal).

Preferably, the antibody (monoclonal or polyclonal, preferably monoclonal, and preferably of isotype IgG and/or IgA) or antibody fragment remains predominantly in soluble form, in the aqueous phase.

Step b)

After step a) of precipitating the raw milk with caprylic acid, the precipitate and the aqueous phase are separated so as to recover the solution comprising the antibody or antibody fragment.

Optionally, step b) of separation, allowing the precipitate to be eliminated, may be further preceded by a step of incubating the mixture, for example for 1 to 4 hours. Said incubation step makes it possible to improve the biological safety of the mixture, in particular by increasing the viral inactivation that occurs. Advantageously, the incubation step also makes it possible to optimize the formation of the precipitate. Advantageously, the separation step is preceded by a step of incubating the mixture for 1 to 4 hours, 1 to 3 hours, or 1 to 2 hours. Advantageously, the separation step is preceded by a step of incubating the mixture equal to 2 hours. During incubation, the mixture may be stirred or not. In a preferred embodiment, during incubation the mixture is not stirred. As a non-limiting example, the incubation step may be performed at room temperature, e.g. between 20 and 25° C.

The separation of the precipitate and the aqueous phase comprising the antibody or antibody fragment can be performed by any separation method known to the skilled person. As a non-limiting example, a conventional liquid/solid separation technique such as draining or mechanical pressing may be used. The separation step can also be performed by centrifugation and/or filtration, for example by tangential filtration, for example by tangential microfiltration, by depth filtration as well as by combinations thereof. Preferably, in the process according to the invention, step b) of separating the precipitate and the aqueous phase comprising the antibody or antibody fragment is performed by depth filtration. In this particular embodiment, the lipids of the cream and the fatty acids such as caprylic acid are advantageously retained by the filter at the same time as the protein precipitate.

“Depth filtration” refers to a filtration process in which the entire filter bed is used to trap particles suspended in the fluid. The fluid thus passes through the filtration bed in its entirety, the particles being trapped on the surface of the filtration bed and in the spaces and/or pores of the filtration bed. Depth filtration may be performed on matrices composed primarily of cellulose fibers, regenerated cellulose, polypropylene, or combinations thereof. These filtration matrices may comprise mineral compounds such as perlite, and/or diatomites, e.g. diatomaceous earth. As an example, the matrix used for the depth filtration may comprise cellulose, propylene, perlite and/or diatomaceous earth.

Preferably, the depth filtration is performed on a matrix composed primarily of cellulose fibers, and preferably comprises perlite, said filter being easily selected by the skilled person. The cut-off threshold of the matrix may be in the range of 10 μm to 80 μm, for example 20 μm to 50 μm. As a non-limiting example, the filter may be a Seitz® T3500, T2600, or T5500 type filter (Pall Corporation). Preferably, the cut-off threshold of said filter is comprised between 10 and 80 μm, preferably between 20 and 50 μm. Preferably, the filter is a 4 to 5 mm thick filter, composed of cellulose fibers and perlite, with a cut-off threshold between 10 and 50 μm, for example of the Seitz® T3500 type.

By “cut-off threshold” is meant herein the diameter of the smallest particle which is 90% retained by the matrix.

In some embodiments, depth filtration is performed in the presence of a filter aid. Thus, according to a preferred embodiment of the invention, the depth filtration is performed in the presence of at least one filter aid (used in alluvialization or as a precoat), which may be mineral (e.g. diatomaceous earth or perlite) or organic (such as cellulose), preferably diatomaceous earth (also called Kieselguhr).

In another embodiment, when the separation step is performed by centrifugation, the skilled person will be able to determine the appropriate centrifugation conditions. As a non-limiting example, the mixture can be centrifuged at 4000×g for 15 min. Preferably, the antibodies or antibody fragments remain predominantly in soluble form, in the aqueous phase located between the cream (on the surface) and the pellet (precipitated proteins). Advantageously, step b) of separation makes it possible to reduce the amount (in g/l) of lipids (in particular of triglycerides) of the composition by at least 20%, preferably by at least 30%, for example by at least 40%, 50%, 60%, 70%, 80%, or even at least 90%, as compared to the initial content (in g/l) of lipids (in particular triglycerides) present in the raw milk before proceeding with steps a) of precipitation and b) of separation. Thus, the composition obtained at the end of step b) advantageously has a lipid concentration (in g/l) that is inferior by at least 20%, preferably at least 30%, for example at least 40%, 50%, 60%, 70%, 80% or at least 90% to that of the raw milk (possibly frozen and then thawed and/or diluted). In particular, the composition obtained after step b) advantageously has a concentration (in g/l) of triglycerides that is inferior by at least 20%, preferably at least 30%, for example at least 40%, 50%, 60%, 70%, 80% or at least 90% to that of the raw milk (possibly frozen and then thawed and/or diluted). As a non-limiting example, lipid content (in g/l) can be measured by colorimetric assay.

Advantageously, at the end of step b) of separation, the amount (in g/l) of proteins (in particular of casein and/or β-lactoglobulin) of the composition is reduced by at least 20%, preferably by at least 30%, for example by at least 40%, 50%, 60%, 70%, 80%, or at least 90% with respect to the initial protein content (in g/l) (in particular casein and/or β-lactoglobulin) present in the raw milk, before the implementation of steps a) of precipitation and b) of separation. Thus, the composition obtained after step b) advantageously has a protein concentration (in g/l) that is inferior by at least 20%, preferably at least 30%, for example at least 40%, 50%, 60%, 70%, 80% or at least 90% to that of the raw milk (possibly frozen and then thawed and/or diluted).

In particular, the composition obtained at the end of step b) advantageously has a concentration (in g/l) of caseins that is inferior by at least 20%, preferably by at least 30%, for example at least 40%, 50%, 60%, 70%, 80% or at least 90% to that of the raw milk (possibly frozen and then thawed and/or diluted). The composition obtained after step b) also advantageously has a concentration (in g/l) of β-lactoglobulin that is inferior by at least 20%, preferably at least 30%, for example at least 40%, 50%, 60%, 70%, 80% or at least 90% to that of the raw milk (possibly frozen and then thawed and/or diluted). As a non-limiting example, protein content (in g/l), such as casein, can be measured by nephelometry and ELISA.

Advantageously, step b) of separation makes it possible to reduce the amount of infectious non-enveloped-type viruses (PPV for example) that may be present in the raw milk by at least 4 logs (decimal logarithms). After steps a) and b) according to the invention, the yield of antibody or antibody fragment is advantageously at least 20%, 30%, 40%, 50%, 60%, 70%, of preferably at least 75% (yield by weight, calculated by comparing the weight of the antibody or antibody fragment in the solution at the end of steps a) and b) to the weight of the antibody or antibody fragment in the milk raw before step a)).

Optional step c)

After step b) of separating the precipitate and the aqueous phase, residual caprylic acid is removed in optional step c) so as to recover the composition comprising the antibody or antibody fragment at high yield and/or purity, advantageously in a form that may be used in therapy. When step b) of separation is performed by centrifugation, this step will also make it possible to eliminate any aggregates that may still be present in the mixture. Preferably, step c) is performed by a depth filtration step performed on a matrix comprising activated carbon, preferably compressed activated carbon. The skilled person knows how to choose a suitable filter according to their general knowledge. As a non-limiting example, the filter that may be used at this stage could be chosen from: the Seitz® AKS5 filter (PALL Corporation), the Seitz® AKS6 filter (PALL Corporation), the R53 SLP filter (3M), the Millistak+® CR40 filter (Millipore), and the Purafix® filter (Filtrox). Preferably, the filter used is the Seitz® AKSS filter (PALL Corporation).

Preferably, at the end of the optional step c) of activated carbon filtration, a composition comprising the antibody or antibody fragment is obtained. Advantageously, at the end of step c) according to the invention, the antibody or antibody fragment yield is advantageously at least 20%, 30%, 40%, 50%, 60%, 65%, preferably at least 70% (yield by weight, calculated by comparing the weight of the antibody or antibody fragment in the solution at the end of steps a) to c) to the weight of the antibody or antibody fragment in the raw milk before step a)).

Preferably, at the end of optional step c) according to the invention, the residual quantity of caprylic acid (expressed as % w/w) is less than 1%, preferably less than 0.5%, even more preferably less than 0.3%, preferably less than 0.1% of the initial amount of caprylic acid (itself expressed in % w/w). Thus, for example, if 1% (w/w) of caprylic acid is added in step a), then the residual amount of caprylic acid at the end of step c) is preferably less than 0.01% (w/w).

Said composition is advantageously suitable for direct administration by the oral or nasal route. By “direct administration” is meant herein that said composition comprising the antibody or antibody fragment does not need to undergo a further step of purification, formulation, concentration, and/or viral elimination, but may be directly administered to a subject or conditioned (e.g., distributed in containers) for future pharmaceutical use comprising an administration by the oral or nasal route.

Said composition comprising the antibody or antibody fragment is preferably stored at about 4° C. (4±2° C.) at the end of the step c) of activated carbon filtration. Indeed, the inventors have shown that the antibodies or antibody fragments contained in the composition are stable for several months at about 4° C. (4±2° C.). It is therefore not necessary to add any excipient, such as a stabilizing agent, at the end of the method of the invention.

Other Optional Subsequent Steps

Although the method described above provides sufficient yield and purity of the antibody or antibody fragment in very few steps, in some cases it may be advantageous to carry out one or more additional steps of purification, concentration, biological safety and/or pharmaceutical packaging. The method according to the invention may thus also comprise at least one additional step, subsequent to step b) of centrifugation or filtration through a depth filter (when step c) is not implemented) or step c) of filtration through an activated carbon depth filter (when this step is performed), chosen from the steps of: concentration (for example by ultrafiltration, tangential ultrafiltration, microfiltration, and/or diafiltration), purification (for example by reverse phase chromatography, hydrophobic interaction chromatography, hydroxyapatite chromatography, cation exchange chromatography, anion exchange chromatography, affinity chromatography, multimodal chromatography, size exclusion chromatography), formulation (for example by addition of components, or by diafiltration), viral safety (for example by solvent-detergent treatment, pasteurization, dry heating, or nanofiltration), and combinations of at least two thereof.

As a non-limiting example, it may be advantageous to concentrate the antibody or antibody fragment comprised in the composition, for example by a step of ultrafiltration and/or diafiltration, in particular in order to obtain a higher concentration of antibody or antibody fragment and/or to formulate the antibody or antibody fragment in a particular composition.

As a non-limiting example, it may also be advantageous to modify the formulation of the antibody or antibody fragment, for example if said antibody or antibody fragment is intended for subsequent parenteral administration, for example by changing the buffer of the composition (notably by diafiltration) and/or by adding at least one pharmaceutically acceptable excipient.

As a non-limiting example, it may also be advantageous to increase the purity of the composition, for example by a chromatography step.

As a non-limiting example, it may also be advantageous to increase the biological safety of the composition when faced with a risk of viral or bacterial infection, for example by a step of sterilizing filtration, by nanofiltration and/or by a step of inactivation (e.g. solvent-detergent treatment, pasteurization, dry heating).

Different combinations of at least two of these additional steps can also be added to steps a) to c) of the method according to the invention.

In a particular embodiment, the method according to the invention further comprises, after step b) of centrifugation or filtration through a depth filter (when step c) is not implemented) or step c) of filtration through an activated carbon depth filter (when this step is performed), one or more of the following steps which aim to adapt the composition to a specific administration and/or to increase the purity of the product:
a concentration step (notably ultrafiltration and/or diafiltration);
a purification step (notably chromatography);
a formulation step; and/or
a step of biological safety

According to a particular aspect, the invention relates to a method of purifying a composition comprising an antibody or antibody fragment from raw milk according to steps a) to b) depth (when step c) is not implemented) or according to steps a) to c) (when this step is performed) above, said method comprising, after step b) of centrifugation or filtration through a depth filter (when step c) is not implemented) or step c) of filtration through an activated carbon depth filter (when this step is implemented), any one of the combinations of the following steps.

Combination 1:
a step of ultrafiltration or diafiltration; and
a formulation step.

Combination 2:
a formulation step;
an ultrafiltration step; and
a step of biological safety.

Combination 3:
a diafiltration step; and
a step of biological safety.

Concentration Step

The concentration step aims to improve the concentration of antibody or antibody fragment in the composition. It may be performed by ultrafiltration and/or diafiltration. When a diafiltration step is used, this may also make it possible to adapt the formulation of the composition.

The concentration step may occur after step b) (when step c) is not implemented) or step c) (when this step is implemented) but before any other step between an additional chromatography step and an additional biological safety step, or after a biological safety step.

Methods and filters suitable for a step of ultrafiltration and/or diafiltration are well-known to the skilled person. Such a step can notably be performed using 30 kDa centramate type cassettes (commercialized by Pall) or 30 kDa Pellicon 2 (commercialized by Merck Millipore) with a dialysis buffer in the case where the ultrafiltration is after a step of viral inactivation and/or viral elimination. As a non-limiting example, the ultrafiltration is a tangential ultrafiltration, e.g. on a membrane having a cut-off threshold of less than 150 kDa.

Purification Step

The purification step is aimed at improving the purity of the antibody or antibody fragment of the composition comprising the antibody or antibody fragment by eliminating various contaminants, such as residual milk proteins or lipids, or possible solvents and/or detergents that may have been used in a previous step. When the composition comprising the antibody or antibody fragment is for therapeutic use and is administered, for example, intravenously, it may be desirable for the composition to be depleted of certain specific antibodies. Thus, when the antibody of interest is a humanized or human chimeric monoclonal antibody (with a human constant region), or when the antibody fragment comprises a constant domain comprising a human Fc fragment binding to human FcR receptors, or comprises a human Fc fragment binding to human FcR receptors, it may be advantageous for the composition to be depleted of endogenous antibodies of the animal. When the antibody of interest is a polyclonal antibody produced by the animal (notably hyperimmunized), it may be advantageous to eliminate certain other antibodies of the animal, notably those having certain antigenic specificities, such as anti-A and/or anti-B antibodies to minimize the risk of hemolysis, directly correlated to the levels of these antibodies.

Although any additional purification step may be used (for example a new precipitation), in the case of an additional purification step, this will preferably be a chromatography step.

Upstream of this additional purification step, the method may also comprise steps intending to modify or adjust the concentration of antibody or antibody fragment of the composition, the conductivity or even the pH of the composition prior to the implementation of the additional purification step.

As a non-limiting example, the additional purification step can be performed by affinity chromatography or ion exchange chromatography, such as anion exchange chromatography or cation exchange chromatography. These techniques are well-known to the skilled person (see, for example, Heegaard, 1998; Hage and Tweed, 1997. In this step, the composition comprising the antibody or antibody fragment from the previous step is applied to the appropriate membrane, which may be chosen by the skilled person in view of their general knowledge.

Preferably, the chromatography step consists of ion exchange chromatography or affinity chromatography, more preferably the chromatography step consists of anion exchange chromatography or cation exchange chromatography.

When the chromatography step is performed by affinity chromatography, the ligand used may be, in a non-limiting manner, a peptide, a microprotein (e.g. 25-200 monomers), a peptidomimetic, a protein, such as protein A, an antibody, an antibody fragment or an aptamer, such as a DNA or RNA aptamer. A suitable aptamer may be selected by the skilled person based on their general knowledge, for example using SELEX technology. Preferably, the aptamer specifically binds IgG and/or IgA antibodies, regardless of the antibody glycosylation profile. More preferably, the aptamer binds at least one IgG subisotype, even more preferably the aptamer according to the invention binds the Fc region of an IgG antibody. Preferably, the aptamer has an IgG dissociation constant of at most 10⁻⁶ M, more preferably from 1×10⁻¹² M to 1×10⁻⁶ M. Preferably, the aptamer binds IgG immunoglobulins at a pH of 5.5. As an example, an aptamer comprising the sequence 5′-CACGGTATAGTCTCGCCA-3′ (SEQ ID NO: 1), 5′-AGGGGCTGGGGTGTGGTTCTGGC-3′ (SEQ ID NO: 2), or 5′-CCCCTAATCAGTGGC-3′ (SEQ ID NO: 3) is particularly preferred. Alternatively, an aptamer comprising a sequence derived from the sequence of SEQ ID NO: 1, 2 or 3 by the deletion, insertion, or substitution of one, two, three, four, or five nucleotide(s) is also particularly preferred.

When a composition depleted of certain antibodies other than the intended antibody or antibody fragment is desired, for example to remove eventual anti-A/anti-B antibodies, affinity chromatography, e.g. as described in application WO 2007/077365 is preferably used.

As a non-limiting example, depending on the type of ligand used, it may be immobilized on the affinity chromatography matrix by Van de Walls forces, or by specific non-covalent interactions. For example, the immobilization of the affinity ligand may depend on a ligand/anti-ligand coupling (e.g. biotin/anti-biotin antibody), or of a marker linked to an aptamer, such as biotin (for binding to avidin or streptavidin), a lectin (for attachment to a sugar moiety), a c-myc marker, a thioredoxin marker, etc. The affinity matrix may be of any type, and is selected according to its use. As an example, the matrix may be a polymeric gel, a filter, or a membrane composed of agarose, cellulose, or one or more synthetic polymers such as polyacrylamide, polyethylene, polyamide, or derivatives of these.

According to a particular embodiment, when the antibody of interest is a humanized or human chimeric monoclonal antibody (with a human constant region), the elimination of residual endogenous antibodies may be performed by affinity chromatography, for example by using an affinity resin capable of selectively retaining the antibody (e.g., wherein the antibody is retained by the antigen it specifically recognizes), the antibody then being recovered by elution, or an affinity resin capable of selectively retaining the endogenous immunoglobulins. Alternatively, when the antibody of interest is a humanized or human chimeric monoclonal antibody (with a human constant region) or when the antibody fragment comprises a human Fc fragment to human FcR receptors, the affinity matrix may comprise a ligand capable of selectively binding endogenous antibodies, which may be an antibody or antibody fragment recognizing the constant region of the antibodies of the non-human animal species used to produce the antibody in its milk.

When the chromatography step is performed by cation exchange chromatography, said chromatography step may be performed, for example, on a resin having as its matrix a crosslinked agarose gel, on which sulfonate groups (—SO₃—) are grafted via dextran-based spacer arms. The conductivity and/or the pH of the composition resulting from the preceding step may preferably be adjusted before application to the resin.

The crosslinked agarose gel, on which sulphonate groups (—SO₃—) are grafted via dextran-based spacer arms used in step c), may preferably be in the form of beads having an average diameter comprised between 10 and 200 μm, preferably between 50 and 150 μm, and notably about 90 μm.

Examples of crosslinked agarose gel matrices on which sulfonate (—SO₃—) groups are grafted via spacer arms include the following matrices: Capto™ S (crosslinked agarose gel matrix on which sulphonate groups (—SO₃—) are grafted via dextran spacer arms in the form of beads with an average diameter of 90 μm, commercialized by GE Healthcare Life Sciences), Fractogel® EMD SO₃⁻ (methacrylate polymer matrix, on which sulfonate groups (—SO₃—) are grafted via long chains of linear acrylamide polymer comprising 15 to 50 acrylamide units, in the form of beads of an average diameter of 30 (type S) or 65 (type M) μm), and Eshmuno®S (crosslinked hydrophilic polyvinylether matrix, on which sulfonate groups (—SO₃—) are grafted via spacer arms, in the form of beads of an average diameter of 75-95 μm). Preferably, the resin having a crosslinked agarose gel matrix on which are grafted is chosen from a crosslinked agarose gel matrix, on which sulphonate groups (—SO₃—) are grafted via dextran-based spacer arms in the form of beads having an average diameter of 90 μm (Capto™ S resin in particular), a methacrylate polymer matrix, on which sulphonate groups (—SO₃—) are grafted via long chains of linear acrylamide polymer comprising 15 to 50 units of acrylamide, in the form of beads having an average diameter of 30 (type S) or 65 (type M) pm (Fractogel® EMD SO₃⁻ resin in particular) and a crosslinked hydrophilic polyvinylether matrix, on which sulphonate groups (—SO₃—) are grafted via spacer arms, in the form of beads with an average diameter of 75-95 μm (Eshmuno®S resin in particular), more preferably the resin is a crosslinked agarose gel matrix, on which sulphonate groups (—SO₃—) are grafted via dextran-based spacer arms in the form of beads with an average diameter of 90 μm (Capto™ S resin in particular).

The elution can notably be performed by increasing the conductivity and/or the pH.

The flow rate of the chromatography step is preferably adjusted to a value corresponding to a residence time comprised between 1 and 3 minutes, preferably between 1.5 and 2.5 minutes and notably of about 2 minutes. Depending on the gel volume, the appropriate flow rate can be calculated on the basis of the following formula: flow rate (mUmin)=gel volume (mL)/residence time (min).

When the chromatography step is performed by anion exchange chromatography, said anion exchange chromatography can be performed, for example, on a hydrophilic polyethersulfone membrane coated with a crosslinked polymer on which quaternary amine groups (Q) are grafted.

This membrane preferably has an average pore size comprised between 0.5 and 1 μm, preferably between 0.6 and 0.9 μm, between 0.7 and 0.9 μm, and notably around 0.8 μm.

The membrane preferably comprises several layers of polyethersulfone coated with a crosslinked polymer on which quaternary amine groups (Q) are grafted, preferably between 10 and 20 layers, notably between 14 and 18 layers, and in particular 16 layers.

An example of such a membrane is the Mustang® Q membrane (hydrophilic membrane of 16 layers of polyethersulfone having an average pore size of 0.8 pm, coated with a crosslinked polymer on which quaternary amine groups (Q) are grafted) commercialized by Pall.

The antibody or antibody fragment composition resulting from the preceding step may be applied to a hydrophilic membrane of polyethersulfone coated with a crosslinked polymer on which quaternary amine groups (Q) are grafted.

The conductivity and/or the pH of the composition comprising the recombinant antibody or antibody fragment resulting from the preceding step may preferably be adjusted before application on the membrane.

Formulation Step

In order to adapt the composition comprising the antibody or antibody fragment obtained by the method according to the invention to a pharmaceutical use, the composition may undergo a formulation step, for example by the addition of an excipient and/or a pharmaceutically acceptable carrier and/or by any other change in the composition of said composition comprising the antibody or antibody fragment, such as a change in buffer. Preferably, the formulation step does not require any specific additional step, but may occur during a diafiltration step, for example when a simple change of buffer is desired. In this case, the diafiltration step can serve both to concentrate and formulate the antibody or antibody fragment. In other cases, the formulation step may be an additional step, separate from the concentration step.

Preferably, during the formulation step, the composition comprising the antibody or antibody fragment will be supplemented with an excipient and/or a pharmaceutically acceptable carrier. In the present description, the term “pharmaceutically acceptable carrier” refers to a compound or combination of compounds used in a pharmaceutical composition that does not cause side effects and that makes it possible, for example, to facilitate the administration of the active compound(s), increase its lifetime and/or efficacy in the body, increase its solubility in solution or improve its conservation. These pharmaceutically acceptable carriers are well-known and will be adapted by the skilled person depending on the nature and type of administration of the selected active compound(s).

The skilled person will be able to choose the excipient(s) to be combined with the antibody or antibody fragment according to the desired galenic form and route of administration. For this purpose, the skilled person may refer to the following references: Pharmaceutical Formulation Development of Peptides and Proteins (S. Frokjaer and L. Hovgaard, Eds., Taylor & Francis, 2000), Remington: The Science and Practice of Pharmacy (Lippincott Williams & Wilkins, Twenty first Edition, 2005) and Handbook of Pharmaceuticals Excipients, American Pharmaceutical Association (Pharmaceutical Press, 6th revised edition, 2009).

The excipient(s) present in the compositions according to the invention may be selected, in a non-limiting manner, from diluents, cryoprotective agents and/or lyoprotectants, stabilizing agents, antioxidants, pH-regulating agents, buffering agents, surfactants, detergents, etc.

Biological Safety Step (e.g. Inactivation and/or Viral Elimination)

To eliminate and/or inactivate viruses and/or other pathogenic macromolecules that would not have been eliminated by precipitation with caprylic acid, such as prions, the agent responsible for transmissible spongiform encephalopathies, and small non-enveloped viruses more resistant to viral inactivation treatments, an additional step of biological safety may be further desired. This step may notably comprise a step of viral inactivation and/or viral elimination, for example by nanofiltration. This step is particularly desirable when the composition obtained by the method described below is intended for parenteral administration, such as via the intravenous, subcutaneous, intradermal or intramuscular route. “Viral inactivation step” refers to a step in which the viruses are not removed from the solution (antigens can still be detected), but are rendered inactive and therefore harmless. These steps notably include dry heating, pasteurization, and solvent-detergent or detergent-only treatment. These various viral inactivation steps are well-known to the skilled person (see, in particular, the WHO guidelines concerning viral inactivation and elimination procedures intended to ensure the viral safety of products derived from human blood plasma, available on the WHO website).

Preferably, in the method according to the invention, the viral inactivation step is a solvent-detergent treatment or treatment with detergent alone. A solvent-detergent treatment is performed by treating the solution with a mixture of solvent, notably tri-(N-butyl)-phosphate (TnBP), and a detergent, notably Polysorbate 80 (polyoxyethylene (20) sorbitan monooleate or polyoxyethylene-p-t-octylphenol (Triton X-100, CAS No. 9002-93-1) under appropriate conditions. An example of a solvent-detergent treatment step is performed in presence of 1% (weight/volume) of Polysorbate 80 and 0.3% (volume/volume) of. The viral inactivation step may also be performed by treatment with a detergent alone, such as Polysorbate 80 (Polyoxyethylene (20) sorbitan monooleate) polyoxyethylene-p-t-octylphenol (Triton X-100, CAS No. 9002-93-1). An example of such a treatment is an incubation for 30 to 120 minutes (in particular for about 1 hour) in a medium comprising 0.5 to 2% (v/v) (notably about 1% v/v) of polyoxyethylene-p-t-octylphenol (Triton X-100, CAS No. 9002-93-1).

By “viral elimination step” is meant a step in which the viruses are removed from the solution, for example, by a nanofiltration step.

As a non-limiting example, the sterilizing filtration may correspond to:
the implementation of one or more steps of sterilizing filtration through filters having a porosity of the order of 0.1 to 0.5 μm (notably of about 0.2 μm, for example with a Millipak filter of 0.22 μm) and/or
through one or more nanofilters of a porosity comprised between 100 and 15 nm, such as 75 nm, 35 nm, 20 nm and/or 15 nm porosity filters, for example
   on filters of decreasing porosity from 100 to 15 nm, in particular on two or three filters arranged in series and having decreasing retention thresholds, for example of 100, 50 and 20 nm, or of 75 and 20 nm, or of 35 and 20 nm, or of 20 and 15 nm
   on filters of the same porosity, in particular on two or three filters arranged in series and having identical retention thresholds, for example of 20 nm or 15 nm.

The most commonly used filters for excluding small non-enveloped viruses, and which can be used in the context of the present invention, are the Planova® filters commercialized by Asahi Kasei, in particular the Planova® 15N and Planova® 20N filters, having an average pore size of 15 and 19 nm, respectively. These filters, constituted of a hollow fiber membrane made of cuprammonium regenerated cellulose, are characterized by a low pore size dispersity (±2 nm around the average size). Alternatively, a Pegasus SV4 filter from Pall or Viresolve® Pro filter (having an asymmetric double polyethersulfone membrane retaining at least 4 decimal logs of virus having a size of at least 20 nm, commercialized by Merck-Millipore) may be used.

Preferably, the nanofiltration step is performed with a filter having a double polyethersulfone membrane with a porosity of about 20 nm. Such filters notably include the Viresolve® Pro filter (having an asymmetric polyethersulfone double membrane with a porosity of approximately 20 nm, commercialized by Merck-Millipore) and the Virosart® CPV filter (having a symmetrical polyethersulfone double membrane of a porosity of about 20 nm, commercialized by Sartorius).

The nanofiltration is preferably performed using a filter having an asymmetric polyethersulfone double membrane with a porosity of approximately 20 nm, such as the Viresolve® Pro filter commercialized by Merck-Millipore. By “a porosity of about 20 nm” is meant that the average pore size of the filter is comprised between 17 and 25 nm, preferably between 17 and 24 nm, between 17 and 23 nm, between 17 and 22 nm, between 17 and 21 nm, 17 to 20 nm, 18 to 25 nm, 18 to 24 nm, 18 to 23 nm, 18 to 22 nm, 18 to 21 nm, 18 to 20 nm, 19 to 25 nm, between 19 and 24 nm, between 19 and 23 nm, between 19 and 22 nm, between 19 and 21 nm, between 19 and 20 nm, between 20 and 25 nm, between 20 and 24 nm, between 20 and 23 nm, between 20 and 22 nm, or between 20 and 21 nm.

In a preferred embodiment, the nanofiltration further comprises a preliminary filtration step through a depth filter comprising cellulose fibers, diatomaceous earth and a negatively charged resin (pre-filter Viresolve PreFilter or VPF) or a polyethersulfone membrane with a porosity of 0.22 μm functionalized by SO₃ groups (pre-filter Viresolve pro Shield in particular).

### EXAMPLES

The invention is illustrated by the following non-limiting examples. These teachings comprise alternatives, modifications, and equivalents as may be appreciated by the skilled person.

### Example 1: Method According to the Invention, Step A) of Purification

The method developed makes it possible to obtain a product of intermediate purity in a single step without any prior step of clarification or delipidation of the raw milk.

Materials and Methods

A series of tests was performed by varying the conditions of step a) of precipitation with caprylic acid according to Table 1 below. In particular, the total protein concentration of the raw milk varied between 6 and 35 g/l, the caprylic acid concentration between 1.3 and 2.4% with regard to the raw milk (w/w), and the pH was adjusted to a value of 4.05 to 5.20 by addition of acetic acid after addition of caprylic acid.

TABLE 1
Operating conditions of the representative tests:

| Test | No 1 | No 2 | No 3 | No 4 | No 5 | No 6 |
|---|---|---|---|---|---|---|
| Total protein concentration | 31 | 35 | 12 | 6 | 9 | 14 |
| (g/l) | | | | | | |
| Caprylic acid (%) | 2.4 | 2.0 | 1.6 | 1.8 | 1.7 | 1.3 |
| Adjusted pH (after addition of | 4.05 | 5.20 | 4.25 | 4.31 | 4.20 | 4.50 |
| caprylic acid and acetic acid) | | | | | | |

Results

As indicated in Table 2 below, the yield and purity of IgG are acceptable when the total protein concentration is comprised between 6 and 14 g/l and the percentage of caprylic acid is comprised between 1.3 and 1.8%. Indeed, the two tests having a protein concentration greater than 30 g/l and a percentage of caprylic acid greater than 2% did not allow steps b) and c) of the method to be performed. However, these two tests were not subject to the same defects. Test no. 1 did not generate a precipitate, whereas Test no. 2 precipitated but the precipitated phase could not be separated from the aqueous phase comprising immunoglobulins in soluble form.

TABLE 2
Operating conditions of the representative tests:

| Test | No 1 | No 2 | No 3 | No 4 | No 5 | No 6 |
|---|---|---|---|---|---|---|
| Yield (IgG, %) | No | Precipitation, | 61 | 40 | 57 | 60 |
| Purity (SDS- | precipi- | no separation | 95 | 93 | 89 | 79 |
| PAGE, %) | tation | of precipitate/ | | | | |
| | | supernatant | | | | |

### Example 2: Method According to the Invention: Step A) of Biological Safety

Following the initial tests, an optimized method was implemented and the various parameters measured at the end of each step in order to determine the effect of each step on a raw milk composition, and more particularly to illustrate the “three-in-one” effect (delipidation, purification, and biological safety) of the precipitation with caprylic acid.

Materials and Methods

Raw goat milk comprising 40 g/l of total protein and 4 g/l of IgG is used as starting material. 130 mL of raw goat milk is diluted 1.22-fold with water. 1% v/v of the X-MLV or PPV virus is added to the diluted raw milk. A sample, referred to herein as “Loading Sample”, is collected. Caprylic acid is added to a final concentration of 1.29% w/w (corresponding to 3.78 g of caprylic acid) and the solution is homogenized for 5 minutes. The pH is adjusted to 4.45±0.05 with acetic acid.

The solution is incubated for two hours at room temperature (22° C.±2° C.) without stirring. A sample, referred to herein as “Hold 1”, is collected.

Alternatively, the pH of the solution is adjusted to 7.0±0.1. A sample, referred to herein as “Hold 2”, is collected.

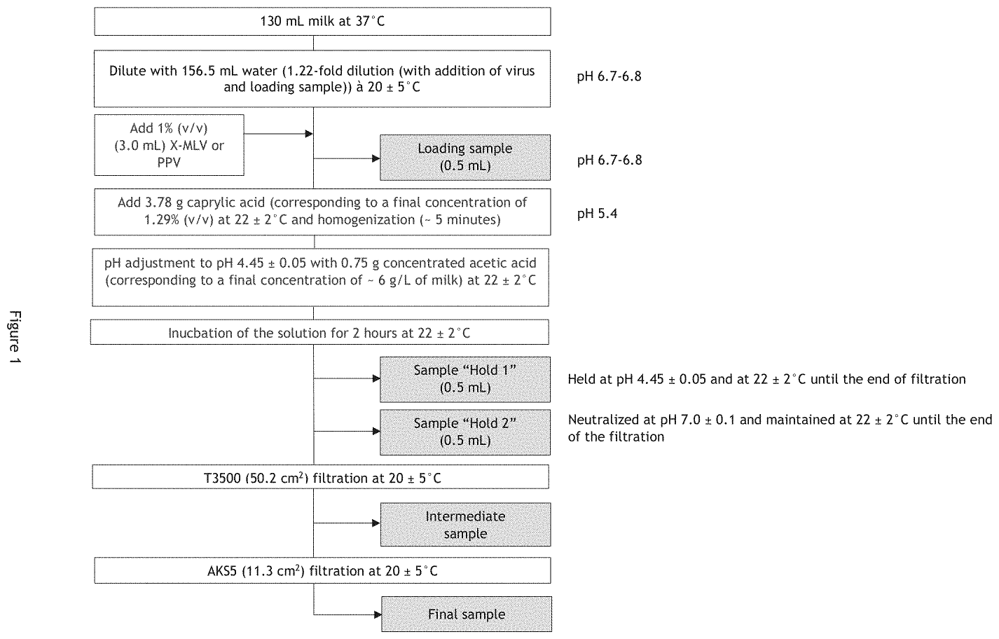

The solution (pH maintained at 4.45) is then subjected to a step of depth filtration using a Seitz T3500 filter (Pall Corporation) at room temperature (i.e. 20° C.±5° C.). A sample, referred to herein as “Intermediate Sample”, is collected. The solution is then subjected to a second step of depth filtration on a charcoal-activated Seitz® AKSS filter (Pall Corporation) at room temperature (i.e. 20° C.±5° C.). This second filtration retains both the filter aids and the remaining caprylic acid, as well as any remaining contaminants, such as milk proteins. At the end of this second filtration step, a sample, referred to herein as “Final sample” is collected. FIG. 1 illustrates this process.

Results

Viral titers were determined for each sample. More particularly, the viral titer was determined before addition of caprylic acid (also referred to as the “Loading” sample), and after the precipitation step (two samples collected, referred to as the “Hold 1” and “Hold 2” samples).

As shown in Table 3 below, the titer of the X-MLV virus (enveloped RNA virus) detected in the “Hold 1” and “Hold 2” samples was reduced compared to the original titer of more than 4.8 decimal logs. The virus is thus inactivated by the operating conditions of step a) (i.e., caprylic acid, pH, duration, temperature).

However, the PPV virus (non-enveloped virus) was not significantly reduced in the Hold 1 and Hold 2 samples. However, the infectious titer detected at the end of step b) shows a reduction in the titer of approximately 4.5 decimal logs, indicating that the operating conditions of step a) (i.e. pH, caprylic acid, duration, temperature) have no effect. In contrast, the reduction of the infectious titer at the end of step b) demonstrates a partitioning effect, this virus being precipitated and physically separated during the separation step.

TABLE 3
Reduction in Viral Titers

| | Reduction factor - | |
|---|---|---|
| | Final filtrate | Comment |
| X-MLV | >4.8 log* | No virus detected in Hold 1 and Hold 2 |
| PPV | 4.5 log* | No significant loss of virus in Hold |
| | | 1 and Hold 2 |

*Logarithmic values expressed in decimal logs.

Although by different mechanisms, the method according to the invention has made it possible to reduce the viral titer by more than 4 decimal logs, both for the enveloped X-MLV virus and for the non-enveloped PPV virus.

### Example 3: Stability of the Composition Comprising the Antibody

The composition obtained by the method was placed at 4° C. for more than 6 months. IgGs thus purified are stable for several months at 4° C.

### Example 4: Method According to the Invention, Step A) of Purification

Materials and Methods

Raw goat or rabbit milk comprising 40 g/l of total protein is used as the starting material.

TABLE 4
Starting material

| | | | Concentration in IgG- | |
|---|---|---|---|---|
| | | | type monoclonal | |
| Sample | Milk | Antibody | antibody | |
| 1 | Transgenic | Anti-TNF transgenic monoclonal | 10-15 | g IgG/L milk |
| | goat | antibody obtained according to | | |
| | | WO2014125374 | | |
| 2 | Transgenic | Anti-HER2 transgenic monoclonal | 70 | g IgG/L milk |
| | goat | antibody obtained according to | | |
| | | WO2014125377 | | |
| 3 | Rabbit | Anti-HER2 monoclonal antibody | 70 | g IgG/L milk |
| | | (trastuzumab) | | |
| 4 | Goat | Monoclonal antibody fragment of | 14 | g Fc/L milk |
| | | the recombinant Fc type fragment | | |
| | | obtained according to | | |
| | | WO2015186004 | | |

130 mL of raw milk is diluted with water. Caprylic acid is added to a final concentration of 1.7% w/w and the solution is homogenized for 5 minutes. The pH is adjusted to 4.3±0.05 with acetic acid for tests 1, 2 and 3 or to 4.4 for test 4.

The solution is incubated for two hours at room temperature (22° C.±2° C.) without stirring and is then subjected to a step of depth filtration using a Seitz T3500 filter (Pall Corporation) at room temperature (i.e. 20° C.±5° C.). The solution is then subjected to a second depth filtration step on a Seitz® AKS5 activated carbon filter (Pall Corporation) at room temperature (i.e. 20° C.±5° C.).

Results

TABLE 5
Yield and purity results

| | Sample | Caprylic acid | | Yield | Purity |
|---|---|---|---|---|---|
| Sample | dilution | concentration (%) | pH | (IgG, %) | (SDS PAGE) |
| 1 | ¼ | 1.7 | 4.3 | 78% | >99.9% |
| 2 | ¼ | 1.7 | 4.3 | 69% | >99.9% |
| 3 | 1/12 | 1.7 | 4.3 | 90% | 87% |
| 4 | ¼ | 1.7 | 4.4 | 80% | 96% |

The results demonstrate that the use of caprylic acid allows for the purification of IgG monoclonal antibodies (samples 1 to 3) with excellent yield and excellent purity (>85%), regardless of the animal origin of the milk and regardless of the IgG monoclonal antibody that is purified. Similarly, the results demonstrate that the use of caprylic acid allows for the purification of monoclonal antibody fragments of the Fc-type fragment (sample 4) with excellent yield and excellent purity (>85%).

### BIBLIOGRAPHIC REFERENCES

Almagro et al. Frontiers in Bioscience 13, 1619-1633 (2008);

Bruggermann et al., Year in Immuno., 7:33 (1993);

Campanella et al., Sensors (Basel). 9(3): 2202-2221 (2009);

Cibelli et al., Science, 280: 1256-1258 (1998);

Dall'Acqua et al., J lmmunol. 169: 5171-80 (2002);

Dall'Acqua et al., J. Biol. Chem. 281:2351-24. (2006) (a);

Dall'Acqua et al. J Immunol; 177:1129-1138 (2006) (b);

Duchosal et al. Nature 355 : 258 (1992);

Edelman et al., Proc. Natl. Acad. USA, 63, 78-85 (1969);

Gene Targeting, A Practical Approach, IRL Press at Oxford University Press (1993);

Gordon et al., Proc Natl Acad Sci USA. 77:7380-4 (1980);

Hage and Tweed, J Chromatogr B Biomed Sci Appl, 699(1-2): 499-525 (1997);

Handbook of Pharmaceuticals Excipients, American Pharmaceutical Association (Pharmaceutical Press; 6th revised edition, 2009);

Hinton et al., J Biol Chem., 279: 6213-6 (2004);

Heegaard, J. Mol. Recognit. 11(1-6): 141-8 (1998);

Hoogenboom et al., J. Mol. Biol., 227: 381 (1991);

Idusogie et al., J lmmunol. 166: 2571-5 (2001);

Jakobovits et al., Proc. Natl. Acad. Sci. USA. 90: 2551 (1993);

Jakobovits et al., Nature, 362: 255-258 (1993) (a);

Jones et al., Nature, 321: 522-525, (1986) (b);

Kabat et al., Sequences of Proteins of Immunological Interest, 5th Ed. Public Health Service, National Institutes of Health, Bethesda, Md. (1991);

Lazar, et al., Proc Natl Acad Sci USA. 103(11): 4005-10 (2006);

Mancini et al., Immunohistochem, 2: 235-254 (1965);

Manipulating the Mouse Embryo, A Laboratory Manual, Second edition, Cold Spring Harbor Laboratory Press (1994);

Marks et al., J. Mol. Biol., 222: 581-5 597 (1991);

Moore et al., mAbs 2:2, 181-189; March/April, (2010);

Pharmaceutical Formulation Development of Peptides and Proteins (S. Frokjaer and L. Hovgaard, Eds., Taylor & Francis 2000);

Remington: The Science and Practice of Pharmacy (Lippincott Williams & Wilkins; Twenty-first Edition, 2005)

Riechmann et al., Nature, 332: 323-327, (1988);

Ryan et al., Science, 278: 873-876 (1997);

Shields et al., J Biol Chem. 276(9): 6591-604 (2001);

Vaughan et al. Nature Biotech, 14: 309 (1996).

Verhoeyn et al., BioEssays, 8: 74, (1988);

Wei et al., Transgenic Res. 20, 321-330 (2011).

1. Method for preparing a composition comprising a monoclonal antibody or a monoclonal antibody fragment from raw milk of a non-human mammal expressing said monoclonal antibody or monoclonal antibody fragment in its milk, comprising:
a) a step of precipitation of the raw milk with caprylic acid,
b) a step of separation consisting of a centrifugation or filtration through a depth filter, and optionally
c) a step of filtration through an activated carbon depth filter.

2. Method according to claim 1, characterized in that step a) makes it possible to both clarify the milk and to render it biologically safe and to purify the monoclonal antibody or monoclonal antibody fragment.

3. Method according to claim 1 or 2, characterized in that step a) precipitates the β-lactoglobulins.

4. Method according to any one of claims 1 to 3, characterized in that the raw milk has not undergone any prior step of clarification and/or skimming and/or acidification.

5. Method according to any one of claims 1 to 4, characterized in that the final percentage (w/w) of caprylic acid used in step a) is comprised between 0.5 and 3.0%, preferably comprised between 1.0 and 2.5%, more preferably between 1.3% and 2.0%, and is preferably 1.7%.

6. Method according to any one of claims 1 to 5, characterized in that in step a) after addition of caprylic acid the pH of the mixture is adjusted to a value less than 4.8, preferably comprised between 4.0 and 4.8, preferably at a value of 4.3.

7. Method according to any one of claims 1 to 6, characterized in that, prior to step a), the raw milk is not diluted or is diluted to a ratio (raw milk:diluent, expressed in volumes) ranging from 1:0.1 to 1:4, preferably 1:3.

8. Method according to any one of claims 1 to 7, characterized in that the total protein concentration of the raw milk before step a) of precipitation with caprylic acid is comprised between 25 and 100 g/l, preferably between 30 and 60 g/l, preferably equal to 50 g/l.

9. Method according to any one of claims 1 to 8, characterized in that the concentration of monoclonal antibody or monoclonal antibody fragment of the raw milk before step a) of precipitation with caprylic acid is comprised between 3 and 50 g/l, preferably between 5 and 30 g/l and preferably equal to 20 g/l.

10. Method according to any one of claims 1 to 9, characterized in that step b) is a depth filtration performed using a filter composed of cellulose fibers.

11. Method according to claim 10, characterized in that said depth filtration performed in step b) is performed using a filter having a cut-off threshold comprised between 10 and 80 μm, preferably between 20 and 50 μm.

12. Method according to any one of claims 1 to 11, characterized in that it further comprises at least one additional step, and subsequent to step b) when step c) is not performed or to step c) when this step is performed, selected from the steps of:
a) concentration, notably by ultrafiltration and/or diafiltration,
b) purification, notably by ion exchange or affinity chromatography, preferably affinity chromatography using aptamer ligands,
c) formulation, and/or
d) biological safety, notably viral inactivation and/or viral elimination.

13. Method according to any one of claims 1 to 12, wherein the non-human mammal expressing a monoclonal antibody or a monoclonal antibody fragment in its milk is a rabbit, a cow or a goat.

14. Method according to any one of claims 1 to 13, wherein the monoclonal antibody fragment is an Fc fragment.
